(19) United States
(12) Patent Application Publication
(10) Pub. No.: US 2006/0286562 A1
(43) Pub. Date: Dec. 21, 2006

(54) Methods for purifying DNA polymerases

(75) Inventors: Ronda M. Allen, Poway, CA (US); Daniel T. McMullan, San Diego, CA (US); Rebecca L. Mullinax, San Diego, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Stratagene California

(21) Appl. No.: 11/283,531

(22) Filed: Nov. 18, 2005

Related U.S. Application Data
(63) Continuation of application No. 09/648,641, filed on Aug. 25, 2000, now abandoned.
(60) Provisional application No. 60/151,805, filed on Aug. 31, 1999.

(51) Int. Cl. C12Q 1/68; C12N 9/22

(52) U.S. Cl. 435/6; 435/199

(57) ABSTRACT

The present invention provides methods and kits for obtaining substantially pure DNA polymerases. The methods comprise fractionating preparations comprising at east one DNA polymerase using Poly U Sepharose chromatography and obtaining substantially pure DNA polymerase. The present invention also provides compositions comprising substantially pure archaebacterial DNA polymerase obtained by fractionation using Poly U Sepharose chromatography resin.

### RELATED APPLICATION INFORMATION

This application claims priority from U.S. Provisional Application Ser. No. 60/151,805, filed Aug. 31, 1999.

### BACKGROUND AND SUMMARY OF THE INVENTION

This invention relates to methods for obtaining substantially pure DNA polymerase. Also provided are compositions of matter comprising substantially purified DNA polymerase and kits for obtaining substantially pure DNA polymerase.

Numerous assays and techniques in the fields of biotechnology and medicine are based on nucleic acid polymerization procedures. The ability to manipulate nucleic acids with polymerization reactions greatly facilitates techniques ranging from gene characterization and molecular cloning (including, but not limited to sequencing, mutagenesis, synthesis, and amplification of DNA), determining allelic variations and single polynucleotide polymorphisms, and detecting and screening for various disease states and conditions (e.g., hepatitis B). DNA polymerases can be used in all of these polymerization techniques, and the activity of polymerases contributes to controlling the sensitivity and reliability of these polymerization reactions.

A common in vitro polymerization technique is polymerase chain reaction (PCR). This process rapidly and exponentially replicates and amplifies nucleic acids of interest. PCR is performed by repeated cycles of denaturing a DNA template, usually by high temperatures, annealing opposing primers to complementary DNA strands, and extending the annealed primers with one or more DNA polymerases. Multiple cycles of PCR result in an exponential amplification of the DNA template.

In the late 1980s, PCR was revolutionized by the use of Thermus aquaticus (Taq) DNA polymerase in place of the Klenow fragment of E. coli DNA polymerase I (Saiki et al., Science 230: 1350-1354 (1988)). The use of the thermostable Taq DNA polymerase obviates the need for repeated enzyme additions during PCR, permits elevated annealing and primer extension temperatures to be employed, and enhances the specificity of PCR. Further, this modification has enhanced the specificity of binding between the primer and its template. But, Taq polymerase has a fundamental limitation in that it lacks a 3′-5′ exonuclease “proof-reading” activity and, therefore, cannot remove mismatched nucleotides added during PCR amplification. Due to this limitation, the fidelity of Taq-PCR reactions have often been less than desirable. Therefore, workers in the field have searched for thermostable polymerases with 3′-5′ exonuclease activity.

Polymerases with 3′-5′ exonuclease activity have been discovered in members of the archaebacteria, also known as the archaea. The archaea are a third kingdom that differs from eukaryotes and bacteria (eubacteria). Many archaea are thermophilic bacteria-like organisms that can grow in extremely high temperatures, i.e., 100° C. Archaebacterial DNA polymerases possess characteristics often not found in their eubacterial, eukaryotic, and bacteriophage counterparts. For example, the archaebacterial DNA polymerases have a markedly high binding affinity for DNA containing uracil (Lasken et al. (J. Biol. Chem. 271: 17692-17696), “Lasken”). Lasken observed that when PCR reactions using archaebacterial DNA polymerases were performed in the presence of deoxyuridine (dUrd)-containing oligonucleotides, DNA synthesis was consistently inhibited. A similar inhibition was not observed by Lasken with bacteriophage, eubacterial (including five thermostable eubacterial enzymes), or mammalian DNA polymerases. Lasken speculated that the inhibition observed with archaebacterial DNA polymerases was due to the formation of a tight, nonproductive complex with dUrd-containing DNA that was not seen with other polymerases.

An archaebacterial DNA polymerase that is particularly useful in PCR reactions is obtained from Pyrococcus furiosus (Pfu). A monomeric DNA polymerase, Pfu DNA polymerase I, that is hyper-thermostable and possesses 3′-5′ exonuclease activity has been identified (Lundberg et al., Gene 108: 1-6 (1991); Cline et al., Nucl. Acids Res. 24: 3546-3551 (1996)). A second heterodimeric DNA polymerase, Pfu DNA polymerase II, has also been identified in Pyrococcus furiosis (European Patent No. EP0870832, published Oct. 14, 1998; Uemori et al., Genes to Cells 2:499-512 (1997)).

In addition to DNA polymerases, DNA replication accessory factors play an important role in the formation of the replication complex that is needed for DNA replication and amplification. Novel accessory factors that enhance the activity of DNA polymerases have previously been identified, produced, purified, and analyzed. See, e.g., International Patent Publication No. WO 98/42860 and U.S. Provisional Patent Application No. 60/146,580 (Pfu Replication Accessory Factors and Methods for Use, Hogrefe et al., filed Jul. 30, 1999). Some of these accessory factors are thermostable homologues of eukaryotic DNA replication proteins such as PCNA, RF-C subunits, RFA, and helicases. Among other accessory factor proteins from archaebacteria that have been analyzed are the PEF (polymerase enhancing factors). PEF have been shown to possess deoxyuracil triphosphatase (dUTPase) activity and are known to affect PCR reactions using hyperthermophilic archaebacterial DNA polymerases.

PCR techniques advantageously should provide sensitive, reproducible results. Reliable thermostable polymerases can help achieve consistent, reproducible results. Accessory factors, in combination with appropriate thermostable polymerases, also help to achieve consistent PCR results. It would be advantageous to establish optimized combinations of thermostable polymerases and accessory factors to provide a more precise, reproducible standard for PCR. Such optimized combinations will greatly improve the reliability and overall results of PCR amplification.

According to certain embodiments, the present invention provides methods to obtain highly purified polymerases. Starting with such highly purified polymerases, i.e., those substantially lacking contaminating proteins and accessory factors, controlled amounts of accessory factors can be added to produce optimized compositions to provide optimal polymerase activity. This optimization process will potentially activate or improve the activities of polymerases, which in turn will improve the results of PCR and other applications that utilize polymerases.

In certain embodiments, the invention provides methods for obtaining substantially pure DNA polymerase comprising fractionation using Poly U Sepharose chromatography.

According to certain embodiments of the inventive methods, the substantially pure DNA polymerase is thermostable polymerase found in members of archaebacteria. In certain embodiments, the substantially pure DNA polymerase is obtained from Pyrococcus furiosus.

In certain embodiments, the invention provides compositions of matter comprising substantially pure DNA polymerase obtained by use of Poly U Sepharose chromatography. In preferred embodiments, the substantially pure DNA polymerase of the inventive composition is a DNA polymerase found in archaebacteria. In certain embodiments, the substantially pure DNA polymerase of the composition is Pfu DNA polymerase I.

In certain embodiments, this invention provides kits for obtaining substantially pure DNA polymerase comprising fractionation using Poly U Sepharose chromatography resin.

It is to be understood that both the foregoing general description and the following detailed description are exemplary and explanatory and are intended to provide further explanation of the invention. The accompanying figures are included to provide a further understanding of the invention. These figures illustrate several embodiments of the invention and, together with the description, serve to explain principles of the invention. The invention is defined by the claims.

### BRIEF DESCRIPTION OF THE DRAWINGS

FIG. 1 is a schematic representation of one embodiment of a DNA polymerase purification scheme comprising fractionation using Poly U Sepaharose 4B chromatography.

FIG. 2 is an SDS-PAGE gradient gel demonstrating purification of Pfu DNA polymerase I, to near homogeneity, using Poly U Sepharose 4B chromatography. Lane 1 contains molecular weight markers using “10 kDa Protein Ladder” from Life Technologies. Those markers include 12 bands in 10 kDa increments (10 kDa to 120 kDa) and one band at 200 kDa (see the faint band at the top of the gel). Lanes 2, 3, and 6 contain Pfu polymerase control samples that were not purified using Poly U chromatography. Lanes 4 and 8 contain 25 units of separate. preparations of Pfu polymerase in a pre-Poly U sample, lanes 5 and 9 contains 25 units of separate preparations of essentially homogenous Pfu polymerase in a post-Poly U sample. Lanes 4 and 5 were obtained from the same preparation. Lanes 8 and 9 were obtained from the same preparation. Lane 7 contained PCR reaction buffer, and no polymerase was known to be present. The apparent molecular weight of Pfu polymerase I, as determined by migration in SDS-PAGE with the “10 kDa Protein Ladder” markers, is approximately 90 kilodaltons. When Pfu polymerase I has been compared to other commercially available markers, it has been reported to migrate at approximately 95 kDa. Lane 10 contains Promega's Pfu polymerase.

FIG. 3 illustrates that the amplification of target in a PCR reaction, using Poly U Sepharose 4B purified Pfu DNA polymerase, is greatly enhanced by the addition of accessory factors. A 3.9 kilobase (kb) human α-1-antitrypsin template was amplified by PCR using appropriate primers. In some reactions, pre-Poly U polymerase samples were used, with and without added PEF. In other reactions post-Poly U polymerase samples were used, with and without added PEF. The amplified products were electrophoresed on an agarose gel. The gel was equilibrated in ethidium bromide and PCR amplification products were visualized. Lane 1 contains molecular weight markers using “Kb DNA Ladder” from Stratagene. The 3.9 kb amplification product is observed in lanes containing the PCR reaction mixture from the pre-Poly U polymerase samples (lanes 7 and 8). No amplification product is seen in the lane containing the PCR reaction mixture from post-Poly U polymerase samples without added PEF (lane 9). When the PCR reaction mixture from post-Poly U polymerase samples is supplemented with PEF, the 3.9 kb amplification product is visualized (lane 10), demonstrating that PEF can be added back to post-Poly U polymerase samples to restore polymerase activity. Lane 2 contains a Pfu polymerase control sample, and lane 3 contains the same control sample as lane 2 with added PEF. Lane 4 contains a second Pfu polymerase control sample, and lane 5 contains the same control sample as lane 4 with added PEF. Lane 6 contains a third Pfu polymerase control sample with added PEF. The data in lane 11 was generated with post Poly U material. That material was obtained from the same pre-Poly U sample that was used to generate the data in lane 7. A larger quantity of that material was run on Poly U with scaled-up procedures when compared to the quantity of material and the Poly U procedures used to obtain the material used to generate the data in lanes 9 and 10. The data in lane 12 was generated with the same material as that used for lane 11 and added PEF.

FIG. 4 illustrates that the amplification of target in a PCR reaction, using Poly U Sepharose 4B purified Pfu DNA polymerase, is greatly enhanced by the addition of accessory factors. A 6.0 kb human α-1-antitrypsin template was amplified by PCR using appropriate primers. In some reactions, pre-Poly U polymerase samples were used, with and without added PEF. In other reactions post-Poly U polymerase samples were used, with and without added PEF. The amplified products were electrophoresed on an agarose gel. The gel was equilibrated in. ethidium bromide and PCR amplification products were visualized. Lane 1 contains molecular weight markers using “Kb DNA Ladder” from Stratagene. The 6.0 kb amplification product is observed in lanes containing the PCR reaction mixture from the pre-Poly U polymerase samples (lanes 7 and 8). No amplification product is seen in the lane containing the PCR reaction mixture from post-Poly U polymerase samples without added PEF (lane 9). When the PCR reaction mixture from post-Poly U polymerase samples is supplemented with PEF, the 6.0 kb amplification product is visualized (lane 10), again demonstrating that when PEF is added back to post-Poly U polymerase samples polymerase activity is restored. Lane 2 contains a Pfu polymerase control sample, and lane 3 contains the same control sample as lane 2 with added PEF. Lane 4 contains a second Pfu polymerase control sample, and lane 5 contains the same control sample as Lane 4 with added PEF. Lane 6 contains a third Pfu polymerase control sample with added PEF.

### DETAILED DESCRIPTION OF EMBODIMENTS OF THE INVENTION

Throughout the specification various documents, including articles, books, patents, and patent applications, are cited. All of these documents are hereby incorporated by reference.

The present invention provides novel methods for obtaining substantially pure DNA polymerase, novel compositions comprising substantially pure DNA polymerase obtained from the novel purification methods, and kits employing the novel methods to obtain the novel compositions of the invention. To facilitate understanding of the invention, a number of terms are defined below.

The term “DNA polymerase” refers to an enzyme capable of catalyzing the template-directed addition of deoxyribonucleotides into a growing DNA polymer. Full-length native forms, as well as fragments, derivatives, and variants that show this template-directed catalytic activity are within the meaning of DNA polymerase, as used herein.

The terms “archaebacterial DNA polymerase” and “archaeal polymerase” refer to DNA polymerases native to members of the archaebacteria, some of which are hyperthermophilic and can survive in extremely high temperatures, i.e., 100° C. Archaebacteria include, but are not limited to, members of the genera Pyrococcus, Thermococcus, Methanococcus, Sulfolobus, Desulfurococcus, and Pyrodictium. There are hyperthermophilic, mesophilic, and thermophilic members of the archaebacteria. Examples of commercially available archaeal polymerases are Pfu polymerase (Stratagene), Vent polymerase (New England Biolabs), Deep Vent polymerase (New England Biolabs), Vent exo (-) polymerase (New England Biolabs), 9°N polymerase (New England Biolabs), and Pwo polymerase (Boehringer Mannheim). All archaeal polymerase fragments, derivatives, and variants with biological activity, that can be used to generate PCR amplification products under appropriate conditions, are within the scope of the present invention. Also contemplated are recombinantly-produced archaeal polymerases that are purified by the novel methods of the invention.

The term “archaeal polymerase fragment,” in contrast to full-length archaeal polymerase, refers to a polypeptide comprising one or more subsets of contiguous amino acids present in an archaeal polymerase. Such a fragment may arise, for example, from a truncation at the amino terminus, a truncation at the carboxy terminus, and/or an internal deletion within the amino acid sequence of the polymerase.

The term “archaeal polymerase derivative” refers to an archaeal polymerase that has been altered so as to contain modified amino acid residues such as norleucine, taurine, etc.

The term “archaeal polymerase variants” refers to archaeal polymerases that have substitutions, deletions, and/or insertions, in the amino acid sequence of a naturally occurring archaeal polymerase. Such “variants” will retain biological activity, as determined by the ability to amplify targets in PCR, and can be purified by the novel methods disclosed in this application. One skilled in the art would appreciate that appropriate changes in the amino acid sequence of a naturally-occurring archaeal polymerase will produce a variant polypeptide that retains biological activity, i.e., the ability to generate amplified product in a PCR reaction under appropriate conditions. Such archaeal polymerase variants are within the intended scope of the claimed invention. Exemplary substitutions are disclosed in U.S. Provisional Patent Application No. 60/146,580 (Pfu Replication Accessory Factors and Methods for Use, Hogrefe et al., filed Jul. 30,1999; now U.S. patent application Ser. No. 09/626,813, Filed Jul. 27, 2000).

One skilled in the art will know that appropriate changes in the amino acid sequence of archaeal polymerases, such as conservative amino acid substitutions, can be made such that biological activity is retained. Conservative amino acid substitutions include, but are not limited to, a change in which a given amino acid may be replaced, for example, by a residue having similar physiochemical characteristics. Examples of such conservative substitutions include, but are not limited to, substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known. See Biochemistry: A Problems Approach, (Wood, W. B., Wilson, J. H., Benbow, R. M., and Hood, L. E., eds.) Benjamin/Cummings Publishing Co., Inc., Menlo Park, Calif. (1981), page 14-15.

The term “substantially pure” refers to polymerase preparations that are at least about 80-85% homogenous, preferably at least about 85-90% homogenous, more preferably at least about 90-95% homogeneous, and most preferably at least about 96%, 97%, 98%, or 99% homogeneous. Homogeneity is determined by analysis of silver-stained SDS-PAGE gels using procedures known in the art.

The term “chromatography” refers to an affinity process, wherein one or more proteins are adsorbed to a suitable chromatography resin or matrix. Examples of suitable matrices include, but are not limited to, ion-exchange resins, hydrophobic resins, dye-binding resins, and the like. Adsorbed proteins are selectively eluted by, for example, linear, concave, convex or step-wise gradients, or the like. Alternatively, the desired protein(s) may not be adsorbed by the matrix and thus will pass through the matrix, while contaminants are adsorbed, and thus removed from the sample. The process may be performed in a column or similar vessel, wherein the sample containing the desired protein(s) are percolated through the column. The use of peristaltic pumps in conjunction with applying the sample to the column, washing the column, and eluting the column is within the scope of the present invention, as is the use of HPLC, FPLC, or similar methodologies. The process also may be performed in a batch process wherein the proteinaceous sample is mixed with suspended matrix material, allowed to adsorb, and then separated by gravity, centrifugal force, or the like.

In certain embodiments of the invention, methods are provided for obtaining substantially pure DNA polymerase using one or more chromatographic procedures. The skilled artisan will appreciate that these chromatographic procedures can generally be performed in different temporal sequences. For example, a hydrophobic chromatography procedure may be performed after a heparin sepharose chromatography procedure. Likewise, a blue sepharose chromatographic procedure may be performed before or after other chromatographic procedures. Further, the skilled artisan will understand that substitution of chromatographic materials with properties similar to particular chromatographic matrices described herein will provide Substantially similar results.

For example, any hydrophobic chromatography matrix may be used, including but not limited to, Octyl Sepharose, Butyl Sepharose, Alkyl Superose, Phenyl Superose, (all from Pharmacia), Methyl Hydrophobic Interaction Chromotography (HIC) resin (BioRad), T-butyl HIC resin (BioRad), TSK-GEL Ether-5PW, Phenyl-5PW, Butyl-NPR (all from Supelco), Toyopearl HIC (TosoHaas), and the like may be used in place of Phenyl Sepharose. Additionally, hydrophobic chromatography matrices other that sepharose may be used, for example agarose-, sephadex-, or acrylamide-based

Further, any affinity matrix may be used. Exemplary dye-binding materials, such as Affi-Gel Blue (BioRad), Cibacron Blue 3 GA (Sigma), and Matrex gel Blue A (Amicon) may be used in place of Blue Sepharose. These materials are all affinity resins.

In lieu of Heparin Sepharose, matrices such as Affi-Gel heparin gel (BioRad), Heparin-5PW (TSK-Gel column, Supelco), Toyopearl AF-Heparin-650M (TosoHaas), and the like may be employed in the invention. These materials are all affinity resins.

Alternatives to Poly U Sepharose 4B include, among others, Polyuridylic Acid-polyacrylhydrazido-agarose (Sigma) as well as numerous uridine-based resins, such as matrices comprising uridine 5′-triphosphate, uridine 5′-diphosphate, and uridine 5′-monophosphate.

A person of ordinary skill will also recognize that adsorbed proteins may be eluted using various gradients. For example, step gradients and concave or convex gradients may be used in place of linear gradients. It will also be apparent to skilled artisans that linear, concave, convex gradients may be run as either an increasing gradient or a decreasing (reverse) gradient. Further, one may employ pH gradients or gradients may comprise a variety of compounds, such as salt, detergent, polyethylene glycol, chaotropic agents, metal ions, biomolecules and/cofactors, such as adenyl-containing cofactors (e.g., NAD⁺) for Blue Sepharose resins, and the like, capable of eluting proteins from the chromatography matrix.

In certain embodiments, the material that is applied to a chromatographic matrix will generally be free of particulate and may have been subjected to additional procedures such as salting-in, salting-out, or the like. Such procedures are designed to assist in keeping a desired protein in solution or to precipitate the desired protein. In certain embodiments, centrifugation is generally employed to separate particulate and insoluble material from solution, but other procedures such as filtration, organic partitioning, or the like, may also be employed.

The skilled artisan will appreciate that a variety of starting materials may be employed in the claimed invention. For example, supernatant fluid from cells that include vectors for expressing secreted forms of polymerase may be employed, obviating the need to disrupt the cells or to remove substantial amounts of particulate and/or cellular debris.

Poly U Sepharose 4B comprises chains of polyuridylic acid that are about 100 U residues in length attached to Sepharose beads. The skilled artisan will appreciate that either shorter or longer chains may be used in the inventive method described in this application. Additionally, the chains of polyuridylic acid may be attached to a resin material or support other than Sepharose. The use of alternatives to polyuridylic acid, as described above, may be useful. One skilled in the art will be able to assess appropriate dimensions and materials for the columns and appropriate conditions for carrying out the chromatography procedures. In the particular embodiment described in the Examples below, the Poly U Sepharose procedure is preceded by certain purification procedures. The skilled artisan will understand that any number of similar or different purification procedures may be used prior to the Poly U chromatography procedure.

One skilled in the art will be able to determine suitable chromatographic processes, for example, as discussed in Deutscher, M. P., Guide to Protein Purification, Academic Press (1990).

A particular embodiment of the invention is described in the following examples. The person of skill in the art will recognize that the poly U chromatography procedure can be many different combinations of purification steps. These examples are offered solely for illustrating the invention, and should not be interpreted as limiting the invention in any way.

### EXAMPLE 1

### Preparation of Soluble, Clarified Cell Extract

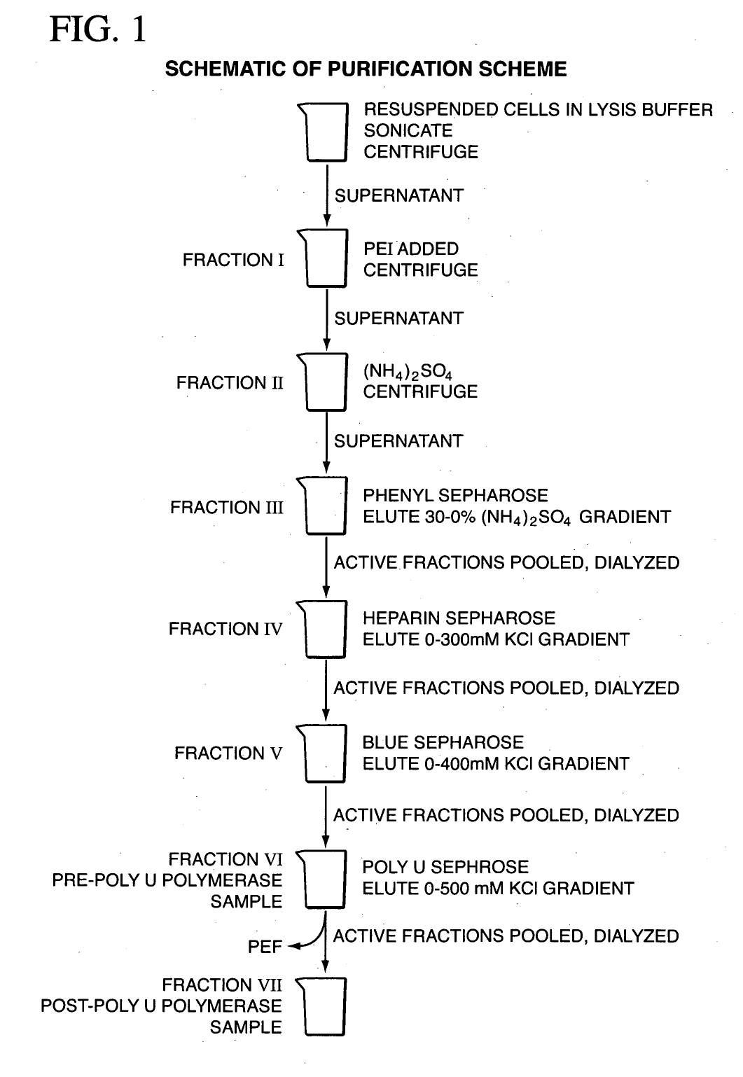

One hundred grams of frozen Pyrococcus furiosis cells were resuspended in four volumes of lysis buffer (50 mM Tris-HCl, pH 8.2, 1 mM EDTA, 1 mM dithiothreitol (DTT), 0.5 mM phenylmethylsulfonyl flouride and 2 mg/ml aprotinin) and disrupted by sonication (using a Bronson Sonifier on setting 8 and duty cycle at 50%, for five two-minute cycles in an ice water bath) and/or by mechanical pressure, such as a French press. The preparation was then centrifuged in a Beckman Ultra LE-80K centrifuge at approximately 29,000×g for 30 minutes to pellet cell debris. The supernatant (Fraction I in FIG. 1) was collected, polyethylenimine (PEI) was added to a final concentration of 0.6%, weight to volume, with stirring and then centrifuged as before. The supernatant (Fraction II) was retained and ammonium sulfate was added to a final concentration of 166 g/l supernatant, with stirring, followed by centrifugation in a Beckman Ultra LE-80 centrifuge at 54,000×g for 30 minutes. The supernatant (Fraction III) was retained.

### EXAMPLE 2

### Chromatographic Purification of Pfu DNA Polymerase I

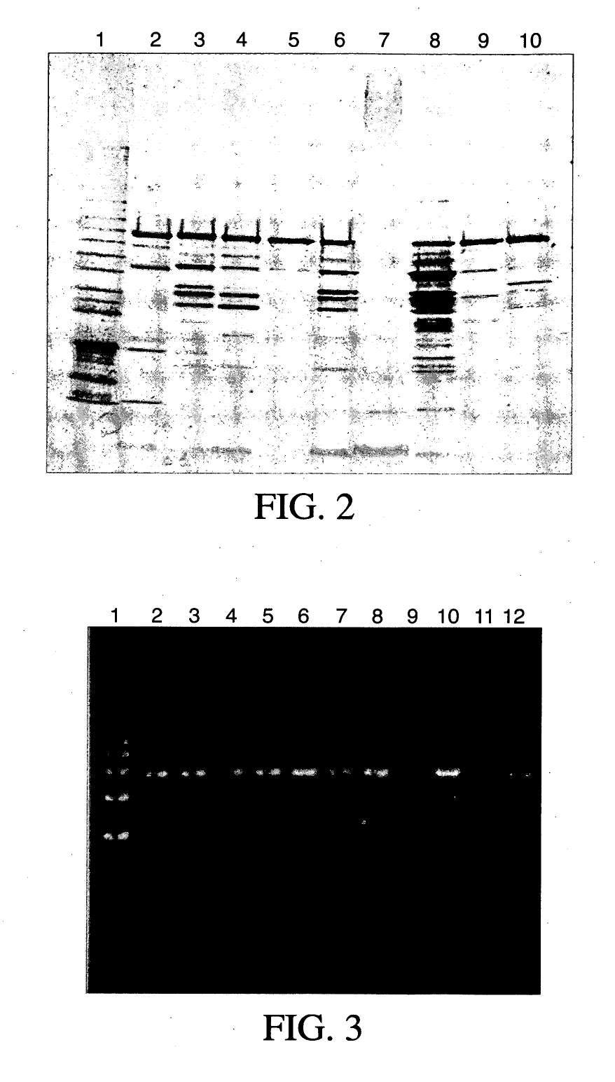

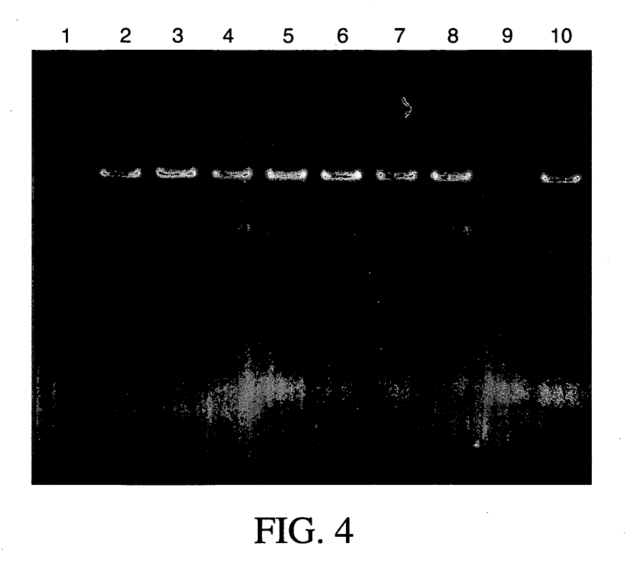

The material ultimately contained in lanes 8 and 9 of FIG. 2, and that ultimately was used to generate the data in lanes 7 to 12 of FIG. 3 and lanes 7 to 10 of FIG. 4, was obtained from a different starting sample than the starting sample used to obtain the material ultimately contained in lanes 4 and 5 of FIG. 2. Those separate sources of material were subjected to the procedures that are discussed in Example 1 and were subjected to the same procedures set forth below except where otherwise noted.

The supernatant (Fraction III) from Example 1 was applied to a 5×5 cm column containing Phenyl Sepharose 6 Fast Flow High Sub® (Pharmacia) equilibrated with 50 mM Tris-HCL, pH 7.5, 1 mM EDTA, 1 mM DTT and 30% ammonium sulfate. The column was operated at a flow rate of 5 ml/minute. The column was washed with 3 column volumes of equilibration buffer. A reverse linear gradient of 30-0% ammonium sulfate in 50 mM Tris-HCL, pH 7.5, 1 mM EDTA, 1 mM DTT (10 column volumes) was used to partition residual PEI, protein contaminants, and the polymerase. Fractions containing peak activity were identified by SDS-PAGE gel analysis (8-16% Tris-glycine acrylaminde gels (Novex) in 25 mM Tris-glycine (pH 8.3), 0.1% SDS; gels were silver stained using methods known in the art, e.g., Deutscher, M. P., Guide to Protein Purification, Academic Press (1990) and/or nucleotide incorporation activity assays (5 μl dilutions of column fractions were added to 45 μl reaction cocktail (50 mM Tris-HCl (pH 8.0), 50 mM KCl, 5 mM MgCl₂, 200 μM each of dATP, dCTP, and dGTP, 195 μM dTTP, 160 μg/ml activated calf thymus DNA, 5 μM ³H-dTTP (NEN, catalog no. NET 221 A), and 1 mM β-mercaptoethanol) and incubated for 30 minutes at 72° C., then quenched on ice. 20 μl of each reaction was spotted on DE81 filters (Whatman), washed seven times with 2×SSC (0.3 M NaCl, 30 mM sodium citrate, pH 7.0), and once with absolute ethanol. Incorporated radioactivity was measured by scintillation counting). Active fractions were pooled and dialyzed against buffer C (50 mM Tris-HCl, pH 8.2, 1 mM EDTA, 1 mM DTT, 10% (v/v) glycerol, 0.1% (v/v) Igepal CA-630, 0.1% (v/v) Tween 20) (Fraction IV).

The dialysate was applied to a 5×5 cm Heparin Sepharose CL-6B® (Pharmacia) chromatography column equilibrated in buffer C. The column was operated at a flow rate of 3 ml/minute. The column was washed with 3 column volumes of equilibration buffer. Polymerase was eluted from the column using a linear gradient of 0-300 mM KCl in buffer C (10 column volumes).

Polymerase-containing fractions, as identified by SDS-PAGE and nucleotide incorporation activity analysis, were pooled and dialyzed against buffer C (Fraction V). This dialysate was applied to a 2.6×3.4 cm (18 ml) column of Blue Sepharose 6 Fast Flow® (Pharmacia) resin, equilibrated in buffer C. The column was operated at a flow rate of 1 ml/minute. The column was washed with 3 column volumes of equilibration buffer at a flow rate of 1 ml/minute. To obtain the material ultimately contained in lanes 8 and 9 of FIG. 2, and that ultimately was used to generate the data in lanes 7 to 12 of FIG. 3 and lanes 7 to 10 of FIG. 4, the polymerase was eluted with a linear gradient of 0-400 mM KCl in a 10 column volume gradient of buffer C with a flow rate of 1 ml/minute. To obtain the material ultimately contained in lanes 4 and 5 of FIG. 2, the polymerase was eluted with a linear gradient of 0-400 mM KCl in a 15 column volume gradient of buffer C with a flow rate of 0.5 ml/minute. The polymerase-containing fractions, identified as before, were pooled and dialyzed against buffer D (50 mM Tris-HCl, pH 8.2, 0.1 mM EDTA, 1 mM DTT, 0.1% (v/v) Igepal CA-630, 0.1% Tween 20, 50% (v/v) glycerol). (Fraction VI, also referred to as pre-Poly U polymerase sample).

Fraction VI performed well when used as a polymerase in PCR. The average yield of polymerase using the method of Examples 1 and 2 was up to ten-fold greater when compared to other purification methods. It was also demonstrated that this method was reproducible and removed inhibitory DNA-binding proteins.

### EXAMPLE 3

### Poly U Sepharose 4B® Chromatography

Materials used to generate the data in lanes 9 and 10 of FIGS. 3 and 4 and the material contained in lane 9 of FIG. 2 were obtained as follows. Fraction VI of Example 2 was further purified using Poly U Sepharose 4B® (Pharmacia). Ten percent of Fraction VI was diluted approximately seven times with buffer C and adsorbed to a column containing Poly U Sepharose 4B (4 ml bed volume; 1×5 cm column) equilibrated in buffer C at 0.3 ml/min. The column was washed with 5 volumes of buffer C and the polymerase was eluted with a 15 column volume linear gradient of 0-0.5 M KCl in buffer C. Fractions containing peak activity, determined as described in Example 2, were pooled and dialyzed against buffer D (Fraction VII, also -referred to as post-Poly U polymerase sample).

Materials used to generate the data in lanes 11 and 12 of FIG. 3 was obtained as follows. Ninety percent of Fraction VI was dialyzed overnight against buffer C (final dialysate volume approximately 50 ml). This dialysate was adsorbed to a column containing Poly U Sepharose 4B (20 ml bed volume; 2.6×3.8 cm column) at a flow rate of 0.5 ml/min. The column was washed with 5 column volumes of buffer C and the polymerase was eluted with a 15 column volume linear gradient of 0-0.5 M KCl in buffer C. Fractions containing peak activity, determined as described in Example 2, were pooled and dialyzed against buffer D (Fraction VII, also referred to as post-Poly U polymerase sample).

The material loaded in lane 5 of FIG. 2 was obtained as follows. Approximately twenty percent of Fraction VI as described for that material in Example 2 was diluted seven times with buffer C and applied to a 2 ml Poly U Sepharose column (1×2.5 cm) at a flow rate of 0.3 ml/min. The column was washed with approximately five column volumes of buffer C and then eluted with a 15 column volume gradient of 0-0.5 M KCl gradient in buffer C.

### EXAMPLE 4

### PCR Analysis of Pre- and Post-Poly U Polymerase Samples

The ability of pre- and post-Poly U polymerase samples to amplify specific targets was evaluated using either a 3.9 kb or a 6 kb human α-1-anti-trypsin gene fragment from human genomic DNA. PCR reactions were performed in the appropriate buffer containing 200 μM of each of the four dNTPs, 100 ng of human genomic DNA, 100 ng of each oligonucleotide primer (3.9 and 6 kb forward primer: 5′-gaggagagcaggaaaggtggaac-3′, SEQ ID NO: 1; 3.9 kb reverse primer: 5′-ttggacagggatgaggaataac-3′, SEQ ID NO: 2; and 6 kb reverse primer: 5′gagcaatggtcaaagtcaacgtcatccacagc-3′ SEQ ID NO: 3), and 2.5 U Pfu DNA polymerase per 50 μl reaction. The buffer used with the 3.9 kb target was 10 mM KCl, 6 mM ammonium sulfate, 20 mM Tris-HCl (pH 8.0), 2 mM MgCl₂ 0.1% Triton X-100, 0.01 mg/ml bovine serum albumin (BSA), while the 6.0 kb target buffer was 20 mM Tris-HCl (pH 8.8), 10 mM KCl, 10 mM ammonium sulfate, 2 mM MgSO₄, 0.1 mg/ml BSA and 0.1% Triton X-100. Some reactions mixtures also contained 1 U PEF. See U.S. Provisional Patent Application No. 60/146,580 (Pfu Replication Accessory Factors and Methods for Use, Hogrefe et al., filed Jul. 30, 1999).

PCR reactions were conducted in 200 μl thin-walled PCR tubes and a PTC-200 DNA Engine (MJ Research, Inc.). Temperature cycle conditions were: 1 cycle at 95° C. for 1 minute, followed by 30 cycles at 95° C. for 30 seconds (denaturation step), 58° C. or 30 seconds (annealing step), and 72° C. for 2 minutes (for 3.9 kb target) or 5 minutes (for 6 kb target) (extension step), and 1 final extension cycle of 72° C. for 4 minutes (for 3.9 kb target) or 5 minutes (for 6 kb target). Five μl of each of the PCR products were analyzed on a 1% agarose/1×TAE (0.04 M Tris-acetate, 0.001 M EDTA) gel for 45 minutes at 80V. The gel was stained with ethidium bromide for approximately 5 minutes by immersing the gel in 1×TAE containing 20 μg/ml ethidium bromide and then the gel was run for an additional 15 minutes at 80V in 1×TAE to destain. The gel was visualized using the Eagle Eye II still video system (Stratagene).

The performance of the pre- and post-Poly U polymerase samples demonstrated that Pfu DNA polymerase is separated from the PEF using the Poly U chromatographic procedure. Little to no PCR amplification products were visualized when Post-Poly U polymerase samples were used in the absence PEF, but with the addition of PEF, amplification products are readily observed.

As shown in FIG. 3, when pre-Poly U polymerase was employed in PCR reactions with appropriate primers and a 3.9 kb human α-1-antitrypsin target, either with or without additional PEF, amplified target is observed. The PCR reaction product from a reaction with pre-Poly U polymerase and no added PEF is shown in lane 7. Lane 8 is the parallel reactions in which PEF was added to the PCR reaction mix. No amplified product is observed in a parallel reaction performed using post-Poly U polymerase without added PEF (lane 9). When PEF is added to the reaction mixture using post-Poly U polymerase, however, amplified product is generated (lane 10).

Similar results are seen in FIG. 4, which shows the reaction products of a PCR reaction performed as described in FIG. 3, except that a 6.0 kb human α-1-antitrypsin target was used. Lane 7 contains samples from a PCR using pre-Poly U polymerase without added PEF; lane 8 contains a sample from a parallel PCR reaction wherein PEF was added. Lane 9 contains a PCR sample from a reaction using post-Poly U polymerase and lane 10 contains a PCR sample from a parallel reaction using post-Poly U polymerase with added PEF. Amplified product is seen in all lanes except those from reactions performed with post-Poly U polymerase without added PEF.

What is claimed is:

1. A method for obtaining DNA polymerase from a sample comprising:
fractionating a sample comprising at least one DNA polymerase using Poly U Sepharose chromatography; and
obtaining substantially pure DNA polymerase.

2. A method of claim 1 wherein the sample fractionated by Poly U Sepharose chromatography is obtained from a prior fractionation of an initial sample comprising at least one DNA polymerase.

3. A method of claim 1 wherein the sample fractionated by Poly U Sepharose chromatography is obtained from a prior chromatography of an initial sample comprising at least one DNA polymerase.

4. A method of claim 3 wherein the prior chromatography comprises hydrophobic chromatography.

5. A method of claim 3 wherein the prior chromatography comprises affinity chromatography.

6. A method of claim 3 wherein the prior chromatography comprises use of a matrix with heparin.

7. A method of claim 6 wherein the prior chromatography comprises use of Heparin Sepharose chromatography.

8. A method of claim 3 wherein the prior chromatography comprises use of a matrix with a dye-binding material.

9. A method of claim 8 wherein the prior chromatography comprises use of Blue Sepharose chromatography.

10. The method of claim 1 wherein the substantially pure DNA polymerase is at least about 95% homogenous.

11. The method of claim 1 wherein the substantially pure DNA polymerase is at least about 85-90% homogenous.

12. The method of claim 1 wherein the substantially pure DNA polymerase is at east about 75-85% homogenous.

13. The method of claim 1 wherein the sample comprises cells that comprise a recombinant expression vector capable of expressing a DNA polymerase.

14. The method of claim 13 wherein the cells are bacterial, yeast, mammalian, or insect cells.

15. The method of claim 1 wherein the sample comprises archaebacterial cells.

16. The method of claim 1 wherein the substantially pure DNA polymerase is an archaebacterial DNA polymerase.

17. The method of claim 1 wherein the substantially pure DNA polymerase is Pfu DNA polymerase I.

18. The method of claim 1 wherein the substantially pure DNA polymerase is Pfu DNA polymerase II.

19. A method for obtaining substantially pure DNA polymerase comprising:
(a) obtaining a sample comprising at least one DNA polymerase;
(b) fractionating the sample using hydrophobic chromatography;
(c) fractionating the product of (b) using Heparin Sepharose chromatography;
(d) fractionating the product of (c) using Blue Sepharose chromatography;
(e) fractionating the product of (c) using Poly U Sepharose chromatography; and
(f) obtaining substantially pure DNA polymerase.

20. A composition of matter comprising a substantially pure DNA polymerase obtained from the method of claim 1 or 19.

21. The composition of claim 20 wherein the DNA polymerase is an archaebacterial DNA polymerase.

22. The composition of claim 20 wherein the DNA polymerase is Pfu DNA polymerase I.

23. The composition of claim 20 wherein the DNA polymerase is Pfu DNA polymerase II.

24. A kit for obtaining substantially pure DNA polymerase comprising poly U chromatography resin.

25. The kit of claim 24 wherein the DNA polymerase is an archaebacterial DNA polymerase.

26. The kit of claim 24 wherein the DNA polymerase is Pfu DNA polymerase.
